(19) United States
(12) Patent Application Publication
(10) Pub. No.: US 2008/0053432 A1
(43) Pub. Date: Mar. 6, 2008

(54) Valve Arrangement

(76) Inventor: Ake Larsson, Jarfalla (SE)

Correspondence Address: SCHIFF HARDIN, LLP;PATENT DEPARTMENT, 6600 SEARS TOWER, CHICAGO, IL, 60606-6473, US

(21) Appl. No.: 11/575,097

(22) PCT Filed: Sep. 1, 2005
(86) PCT No.: PCT/EP05/54293

§ 371 Date: Mar. 12, 2007

(30) Foreign Application Priority Data
Sep. 15, 2004 (SE) 0402217-4

(51) Int. Cl. A61M 16/10

(52) U.S. Cl. 128/200.24

(57) ABSTRACT

A valve arrangement has a valve housing with an inlet and an outlet and a flow regulator including a porous body that is displaceable in the valve housing between the inlet and the outlet. The porous body blocks flow between the inlet and the outlet except for flow through the porous body. The displacement of the porous body between the inlet and the outlet proportionally varies a size of an outer surface of the porous body that is exposed to the outlet, thereby regulating fluid flow between the inlet and the outlet.

### BACKGROUND OF THE INVENTION

1. Field of the Invention

The invention relates to a valve arrangement of the type having a valve housing with an inlet and an outlet and with a flow regulator which is arranged in the housing so that a movement of the housing relative to the flow regulator can be performed, so a proportional variation of the flow is achieved.

2. Description of the Prior Art

In U.S. Pat. No. 5,845,633, a valve arrangement of the above type is described. The object of that valve arrangement is to dose small amounts of nitric oxide to a breathing gas, the gas being supplied to a patient for medical purposes. The valve housing is here a tube-shaped container in which the flow regulator in the form of a tube-shaped membrane is arranged, with the container and the end side of the membrane which being turned towards the breathing gas being made of a material which does not allow nitric oxide to pass through. The membrane is connected via a gas tube with a source for nitric oxide. The tube-shaped membrane is made using a material which allows nitric oxide through, e.g. TeFlon®. The tube-shaped membrane is also removably arranged in the container. The nitric oxide source is suitably regulated so that a constant pressure is prevalent in the membrane tube. This results in a constant difference in partial pressure for nitrogen oxide on the in- and out-side of the membrane tube. Diffusion from the inside of the membrane tube to the breathing gas, which depends on the size of the membrane tube's diffusion surface, is obtained. The dosing is regulated by exposing a suitable portion of the total diffusion surface. The exposure of the diffusion surface occurs by taking a suitable part of the surface out of the container. This described ventilator arrangement is only related to dosing of nitrogen oxide.

### SUMMARY OF THE INVENTION

An object of the invention is to provide a valve arrangement of the type described above which is related to regulating the micro-flow for different sorts of gases as well as different sorts of liquids.

The above object is achieved in accordance with the invention by a flow regulator having a porous body to block the flow between the inlet and the outlet except for through the porous body wherein, through relative movement, a progressive variation of an outer surface of the porous body is obtained, the outer surface being in flow-contact with the outlet. By organizing the valve according to the invention, and particularly because of the porous body through which a gas or a liquid can pass, a universal valve is obtained for micro-flows, that allows such flows can to be easily be dosed.

In an embodiment of the valve arrangement according to the invention, the valve housing has a first part containing the inlet, the first part having a sealing surface towards which at least a part of the porous body's outer surface lies, and a second part having the outlet, wherein the second part's inner diameter is larger than the body's outer diameter, and the relative movement is a displacement of the body between the first and the second part. By a gradual displacement of the porous body within the second part of the valve housing, the dosing of gas or liquid is increased.

In another further embodiment of the valve arrangement according to the present invention, the porous body has a first non-porous end part, which is turned towards the second part of the ventilator housing, and that the first part is dimensioned so that the non-porous end part can be placed in the first part of the ventilator housing. In this way, when the porous body has been displaced to a location where the non-porous end part is placed in the first part of the valve housing, the body's entire outer surface lies towards the sealing surface in the first part, so the valve arrangement is closed.

In Another embodiment of the valve arrangement according to the invention, the porous body is provided with at least one channel, the opening of which lies in the body's second end part which is turned towards the inlet. In this way gas or liquid can more quickly reach the porous body's interior and so have shorter routes through the pores to the outer surface of the body.

### DESCRIPTION OF THE DRAWINGS

FIG. 1 is a longitudinal section through a valve arrangement according to the invention, in a closed position.

FIG. 2 shows the valve arrangement of FIG. 1 in a partially opened position.

FIG. 3 shows the valve arrangement of FIGS. 1 and 2 in a completely opened position.

FIG. 4 is a longitudinal section through a further embodiment of a valve arrangement according to the present invention.

### DESCRIPTION OF THE PREFERRED EMBODIMENTS

In FIG. 1, a valve arrangement 1 is shown in longitudinal section which is designed to regulate micro-flows for different sorts of gases. The valve arrangement 1 has a valve housing 2 with an inlet 3, one or more outlets 4 and a flow regulator 5 in the form of a porous body which is displaceably arranged in the housing 2 along a central axis 8.

With this valve arrangement, gas can flow through the inlet 3 into the valve housing 2 and to reach the porous body 5. The gas then passes over pores 20 in the body 5 to the outer surface 9 of the body 5, so gas can be emitted through the outlets 4. This will be described in detail below.

The valve housing 2 can be divided into a first part 6 and a second part. The inlet 3 is arranged in the first part 6. The inner diameter of the first part 6 of the housing 2 is dimensioned so that the outer surface 9 of the porous body 5 lies tight to the inner wall 10 of the housing 2, the inner wall 10 here serves serving as a sealing surface.

In the second part 7 of the ventilator housing 2, the outlets 4 are arranged. The inner diameter of the second part 7 is larger than the outer diameter of the porous body 5. The porous body 5 has a first non-porous end part 11, which is turned towards the second part 7 of the housing 2. The porous body 5 is provided with a channel 12, the opening of which lies in the second end part 14 of the porous body 5, which end part 14 is turned towards the inlet 3.

The end side 16 of the second part 7 of the housing 2 is provided with a through-passage opening 17, through which an actuator 15 in the form of a straight peg extends, the opening 17 preferably being arranged so that the actuator 15 is displaceable along the central axis 8.

A spring 19 is placed between the second end part 14 of the porous body 5 and the inner wall 18 in the first part 6 of the housing 2, which is turned towards the end part 14.

In FIG. 1, the porous body 5, with help of the actuator 15 pushing towards the body's non-porous end part 11, has been displaced in the ventilator housing 2 along the central axis 8 so that the entire body 5 is placed in the first part 6 of the housing 2, the spring 19 in this position being compressed. Because of the inner wall 10 that seals against the entire jacket surface 9 of the body 5, and because of the first non-porous end part 11 of the body 5 which has an axial sealing function, the valve in this position is closed

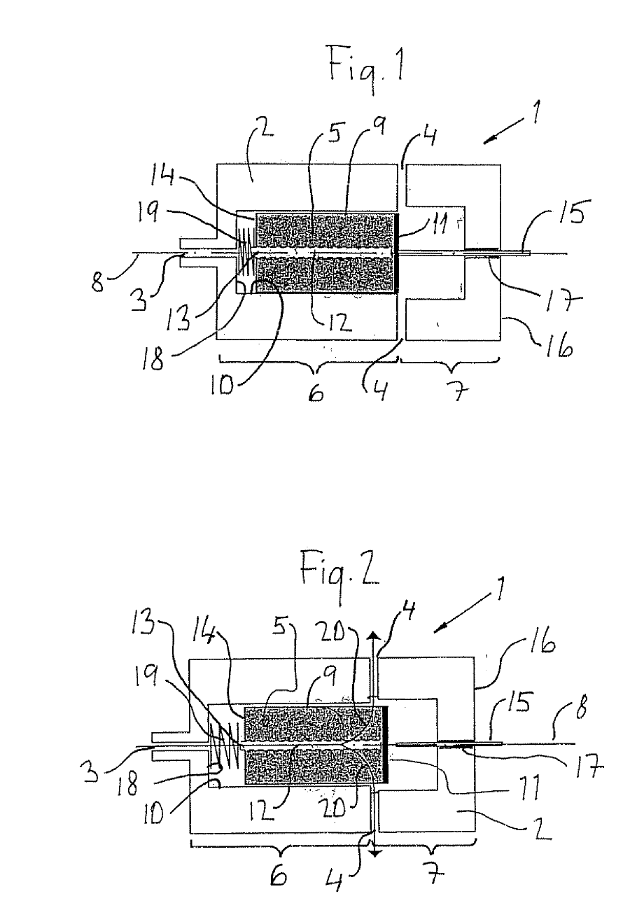

In FIG. 2, it is shown that the porous body 5 by the action of the spring 19 and the actuator 15 has been displaced in the direction towards the second part 7 of the housing 2, so a part of the jacket surface 9 of the body 5 has been exposed between, as earlier described, the inner diameter of the second part 7 of the housing 2 is larger than the outer diameter of the body 5. In this way, gas comes from pores 20 in the body 5 which results in the exposed jacket surface 9 being in flow-contact with the outlets 4.

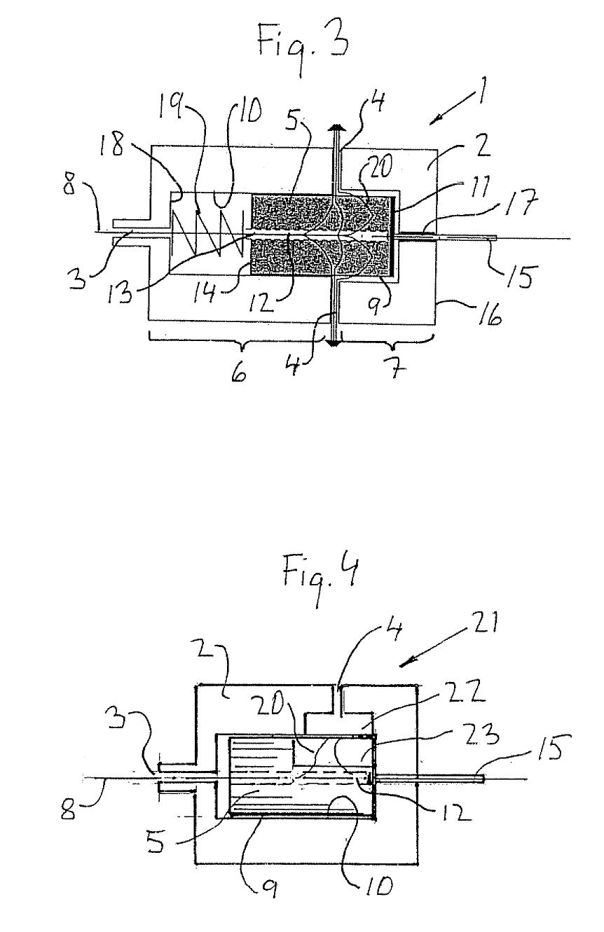

In FIG. 3, it is shown that the porous body 5 has been displaced to a position where the valve arrangement 1 is completely open. In this position a maximum jacket surface 9 of the body 5 is exposed. In this way, a maximum number of pores 20, resulting in the exposed jacket surface 9 of the body, have flow-contact with the outlets 4. Only a few pores 20 have been depicted in the Figures.

The channel 12 described in connection with FIG. 1 serves to easily and quickly distribute the gas in the porous body 5.

In FIG. 4, a further example of a valve arrangement 21 for regulating micro-flows for gases is shown in longitudinal section. In the description of this valve arrangement 21, the same reference numerals as in FIGS. 1-3 have been used as much as possible.

The valve arrangement 21 has a valve housing 2 with an inlet 3 and an outlet 4, and a flow regulator 5 in the form of a porous body. The interior of the housing 2 is dimensioned so that the body 5 can only rotate around the central axis 8 by the action of the actuator 15, which is solidly fixed to the body 5. The outer surface 9 of the body 5 is for the most part provided with a coating which serves as sealing layer for gases. Only a smaller surface 23 is exposed. The inner wall 10 of the housing 2 has in connection with the outlet 4 a recess 22 which extends along part of the outer surface of the body 5. By turning the body 5 so that the surface 23 is in front of or partially in front of the recess 22, the pores 20 resulting in this surface have more or less flow-contact with the outlet 4. When the body 5 has been turned to a position where the sealing layer completely covers the recess 22, the valve arrangement 21 is closed. Even this valve arrangement 21 can preferably be provided with a channel 12 in the porous body 5, the benefits of which have been described above.

Due to the relative movement between the housing 2 and the porous body 5, described in connection with FIGS. 2, 3 and 4, a progressive variation of the outer surface of the porous body 5 is achieved, which surface is in flow-contact with the outlets 4, so an extremely careful micro-dosing of gas can be achieved.

The valve arrangements according to the invention described herein can also regulate micro-flows for liquids.

The valve arrangements described can also be used in connection with anaesthetic systems. In this way, an anaesthetic liquid can be supplied to the valve housing, the liquid being vaporized in the porous body, with the body preferably being heated so that the liquid will more easily be vaporized.

Although modifications and changes may be suggested by those skilled in the art, it is the invention of the inventor to embody within the patent warranted heron all changes and modifications as reasonably and properly come within the scope of his contribution to the art.

I claim as my invention:

1-4. (canceled)

5. A valve arrangement comprising:
a valve housing comprising a first part containing an inlet and a second part containing an outlet;
a flow regulator in said valve housing, said flow regulator comprising a porous body that is displaceable between said first part and said second part, said porous body blocking fluid flow between said inlet and said outlet except for fluid flow through said porous body, and said displacement of said porous body between said inlet and said outlet progressively varying a size of an outer surface of said porous body exposed to said outlet; and
said first part of said valve housing comprising a sealing surface facing toward at least a part of said outer surface of said porous body, and said second part having an inner diameter that is larger than an outer diameter of said porous body.

6. A valve arrangement as claimed in claim 5 wherein said porous body comprises a non-porous end part facing toward said second part of said housing, said non-porous end part having a size and shape allowing said non-porous end part to fit into said first part of said valve housing.

7. A valve arrangement as claimed in claim 6 wherein said non-porous end part is a first end part, and wherein said porous body comprises a second end part and at least one channel opening into said second end part, said second end part facing said inlet.
